(19) United States
(12) Patent Application Publication
(10) Pub. No.: US 2003/0105054 A1
(43) Pub. Date: Jun. 5, 2003

(54) GPI-anchored cytokines

(75) Inventors: Thomas E. Wagner, Greer, SC (US); Yanzhang Wei, Greenville, SC (US)

Correspondence Address: FOLEY AND LARDNER, SUITE 500, 3000 K STREET NW, WASHINGTON, DC, 20007, US

(73) Assignee: Greenville Hospital System

(21) Appl. No.: 10/228,262

(22) Filed: Aug. 27, 2002

Related U.S. Application Data
(60) Provisional application No. 60/314,653, filed on Aug. 27, 2001.

(51) Int. Cl. A61K 48/00; A61K 38/20

(52) U.S. Cl. 514/44; 424/93.2; 424/85.2

(57) ABSTRACT

The present invention relates to immunogenic compositions for stimulating T cell proliferation and methods for enhancing therapeutic effectiveness of some traditional anti-cancer treatments. Specifically, local delivery of cytokines that target the plasma membrane of a cancerous cell exhibit more potent anti-tumor effects than systemic delivery of cytokines in soluble form.

### CROSS-REFERENCE TO RELATED PATENT APPLICATIONS

This application claims priority to U.S. Provisional application serial No. 60/314,653, filed Aug. 27, 2001.

### BACKGROUND OF THE INVENTION

1. Field of the Invention

The present invention relates to compositions and methods for eliciting an immune response and enhancing current therapeutic modalities for the treatment of cancer. Specifically, the invention relates to making an immunogenic composition that is directed to a cell surface and stimulates T cell proliferation.

2. Description of Related Art

Attempts have been made during the past two decades to develop immunotherapies for treatment of cancer based on stimulating the host immune response to the tumor. These approaches were based on attempts to immunize against specific tumor cells or with nonspecific stimulants in the hope that general immune stimulation would concomitantly increase the host anti-tumor response. Some experimental evidence indicated that this approach might be feasible in the therapy of established tumors. However, the inability to stimulate sufficiently strong responses to putative tumor antigens and the general immunoincompetence of the tumor bearing host, were factors that argued against the success of this approach.

Nevertheless, attention has focused on the use of cytokines in an attempt to augment the immune response to tumor-associated antigens in recent years. Cytokines such as interleukin 2 (IL-2) or interferon (IFN-.gamma.) have been used to treat neoplastic disease with marginal therapeutic impact. Vieweg et al. (1995) Cancer Investigation 132(2):193-201. Cytokines do not exhibit direct toxic effect on cancer cells; their anti-tumor activity is mediated by modulation of the host's immunological response to the neoplasm. For example, interferon-.gamma. induces the expression of MHC class I determinants and augments the sensitivity of tumor cells to cytotoxic T cell-mediated lysis. Lichtor et al. (1995) J. Neurosurg 83:1038-1044. IL-2 is required for the growth of cytotoxic T lymphocytes and enhances natural killer (NK) and lymphokine-activated killer cells (LAK). The limited effect of systemic administration of IL-2 in cancer immunotherapy has been partially explained by the short half-life of IL-2 and severe toxicity due to necessary high doses. Vieweg et al. (1995). Moreover, the severe side effects caused directly or indirectly by IL-2 has been an obstacle to the development of routine treatment protocols based on the approach, e.g. Gaynor et al, Ann. Int. Med., Vol. 109, pgs. 953-958 (1988); Lee et al, J. Clin. Oncol., Vol. 7, pgs. 7-20 (1989); and Rosenberg et al, Human Path., Vol. 22, pgs. 493-502 (1990).

Lymphokine-activated killer cells (LAK) have also been used as an approach to elicit a cellular immune response. LAK cells are MHC-unrestricted lymphoid cells which kill fresh tumor cells but not normal cells. Tumor-infiltrating lymphocytes (TIL) are predominantly MHC-restricted T cells which have been found to be 50-100 times more potent than LAK cells in murine models. The use of LAK or TIL either alone or with IL-2 has shown some anti-tumor effects. In the combined approach however, IL-2 toxicity remains a problem. Vieweg et al. (1995).

A major advance in cancer treatment would be made if therapeutic methods could reduce the severity of side effects directly and indirectly caused by IL-2. The side effects caused, in part, by a high dose systemic injection. Local or regional treatment instead of systemic application is an alternative approach to eliciting a larger immune response and fewer side effects. Additionally, immobilizing IL-2 or other cytokines on the tumor cell may enhance effectiveness of current cancer treatments.

A wide range of cell-surface proteins, including enzymes, coat proteins, surface antigens, and adhesion molecules, are attached to the plasma membrane via GPI anchors (Burikofer et al. FASEB J. 15:545 (2002)). GPI is a posttranslationally added lipid anchor; therefore, unlike conventional polypeptide anchors which have different transmembrane domains and connect to specific cytoplasmic extensions, GPI anchors use a common lipid structure to attach to the membrane, which is irrespective of the proteins linked with it (Englund et al., Annu. Rev. Biochem. 62:121 (1993)). GPI anchor signal sequences have been identified for many proteins such as decay accelerating factor (DAF) and leukocyte function antigen-3 (LFA-3) (Caras et al., Science 243:1196 (1989)). The GPI anchor signals have been successfully engineered onto the C-terminus of other un-GPI anchored proteins, and these GPI anchored proteins are coated on the cell surface and are functional. (Anderson et al., P.N.A.S. 93:5894 (1996); Brunschwig et al., J. Immunother. 22:390 (1999)). Therefore, GPI anchor is a very useful technology to engineer proteins onto the cell surface.

### SUMMARY OF THE INVENTION

It is therefore an object of the present invention to overcome the obstacles afforded by traditional cancer therapies and provide compositions and methods that generate a greater immune response with fewer side effects than other anti-tumor treatments.

To this end, an immunogenic composition containing a vector comprising a nucleic acid encoding a factor that stimulates T cell proliferation attached to a sequence that signals a GPI anchor is described. It is preferred that the vector of the immunogenic composition is a plasmid or a virus vector. Preferably, the virus is a conditionally replicating adenovirus. Also preferred, the factor that stimulates T cell proliferation is a cytokine. Still preferred, the cytokine is IL-2 or IL-12. In a preferred embodiment, the vector comprises a nucleic acid encoding human IL-2 attached to a GPI anchor sequence of decay accelerating factor (DAF).

Similarly, a pharmaceutical composition comprising the immunogenic composition containing a vector comprising a nucleic acid encoding a factor that stimulates T cell proliferation attached to a sequence that signals a GPI anchor and a pharmaceutically suitable excipient is described. Preferably, the factor that stimulates T cell proliferation is a cytokine and still preferred, the cytokine is IL-2 or IL-12.

The instant invention also contemplates an immunogenic composition comprising a factor that stimulates T cell proliferation and a GPI anchor as well as an immunogenic composition comprising a factor that stimulates T cell proliferation attached to the plasma membrane of a cell via a GPI anchor, wherein said cell is a cancer cell. Preferably, the cancer cell is a melanoma cell. A pharmaceutical composition containing these immunogenic compositions and additionally a pharmaceutically suitable excipient is also addressed. In a preferred embodiment, the factor that stimulates T cell proliferation is a cytokine. Still preferred, the factor is IL-2 or IL-12.

Furthermore, a method of making an immunogenic composition containing a vector comprising modifying a nucleic acid encoding a factor that stimulates T cell proliferation to include a sequence that signals a GPI anchor is described. It is preferred that the vector is a plasmid or virus vector. Preferably, the virus is a conditionally replicating adenovirus. Also preferred, the factor that stimulates T cell proliferation is a cytokine. More preferred, the cytokine is IL-2 or IL-12.

According to another aspect of the present invention, therefore, is a method of eliciting an immunogenic response, comprising contacting a target cell with an immunogenic composition containing a vector comprising a nucleic acid encoding a factor that stimulates T cell proliferation attached to a sequence that signals a GPI anchor. Preferably, the target cell is a cancer cell and more preferably, the target cell is a melanoma cell. Also preferred, the factor is a cytokine, specifically IL-2 or IL-12. The method can contain a vector that is a virus or a plasmid. Preferably, the virus is a conditionally replicating adenovirus.

In yet another aspect, the present invention provides a method of treating a patient comprising administering a therapeutically effective amount of a pharmaceutical composition comprising an immunogenic composition containing a vector comprising a nucleic acid encoding a factor that stimulates T cell proliferation attached to a sequence that signals a GPI anchor and a pharmaceutically suitable excipient. It is preferred that the vector is a plasmid or a virus vector. Preferably, the virus is a conditionally replicating adenovirus. Also preferred, the factor that stimulates T cell proliferation is a cytokine. Still preferred, the cytokine is IL-2 or IL-12.

It is another object of the present invention to provide a method for preparing a cancer vaccine comprising (i) preparing a feeder layer of cells that express a factor that stimulates T cell proliferation on their plasma membrane, (ii) exposing a cancer cell or cancer cell hybrid to said feeder layer, (iii) optionally irradiating said cancer cell or said hybrid and (iv) administering said exposed cancer cell or said hybrid to a patient. The factor as described herein is preferably a cytokine. More preferably, the cytokine is IL-2 or IL-12. Also preferred, the cancer cell is a melanoma cell. Still preferred, the hybrid cell is a fusion between a cancer cell and a dendritic cell.

### BRIEF DESCRIPTION OF THE DRAWINGS

FIGS. 1A-1D. FIGS. 1A-1D are tables comparing number of tumor nodules in untreated tumor cells, tumor cells treated with soluble IL-2 or tumor cells treated with GPI anchored IL-2.

FIGS. 2A-2C. Pulmonary metastasis images comparing untreated tumor cells with tumor cells treated with soluble IL-2 and tumor cells treated with GPI anchored IL-2. 2×10⁵ B16F0 cells, B16F0/IL2s cells, or B16F0IL2gpi cells were intravenously injected into female C57BL/6J mice. Four weeks later, the mice were sacrificed and the tumor nodules on the lungs were counted and photographed. A, Lungs from B16F0 cell injected mice. B, Lungs from B16F0/IL2s cell injected mice. C, Lungs from B16F0/IL2gpi cell injected mice.

FIGS. 3A-3C. GPI anchored IL-2 expression. A, Cell surface IL-2 was monitored by FACS after rat anti-human IL-2-FITC staining of B16F0 cells (-----), B16F0/IL2gpi cells after treatment of PI-PLC ( [symbol] ), or B16F0/IL2gpi cells ( [symbol] ). B, Membrane bound IL-2 was harvested from 1×10⁷ B16F0 cells, B16F0/IL2s cells and B16F0/IL2gpi cells by PI-PLC treatment and measured with the human IL-2 ELISA kit. * * * , P<0.001. C, IL-2 activity in the culture medium of B16F0 cells, B16F0/IL2s cells, or B16F0/IL2gpi cells.

FIGS. 4A-4F. Immunohistochemical analysis of tumors developed from B16F0 cells (A, B), B16F0/IL2s cells (C, D), or B16F0IL2gpi cells (E, F). 1×10⁵ of the above cells were subcutaneously injected into female C57BL/6J mice. Ten days later, the tumors were recovered and frozen sections were made. After fixation, slides were first stained with rat anti-mouse CD4 (A, C, E) or rat anti-mouse CD8 (B, D, F). The slides were then stained with goat-anti-rat IgG conjugated with horse reddish peroxidase (HRP) and developed using Vectastain Elite ABC immunohistochemical kit.

FIGS. 5A-5D. Local high levels of IL-2 achieved by GPI anchored IL-2. Tumor cells isolated from two weeks old subcutaneous tumors were used to make slides by Cytospin. The slides were immunohistochemically stained for IL-2. A. Tumor cells from regular B16F0 tumor; B. tumor cells from B16F0/IL2s tumor; C. tumor cells from B16F0/IL2gpi tumor; D. Tumor cells were treated with PI-PLC and the IL-2 level of the supernatant were measured by ELISA. Data are the average of 4 replicates. * * * : p<0.001.

### DETAILED DESCRIPTION OF THE PREFERRED EMBODIMENTS

The present inventors have discovered compositions and methods that elicit a strong immune response against tumor cells with fewer side effects than conventional treatments. The invention is achieved by linking certain therapeutic molecules to the surface of a target cell.

More specifically, the present inventors have developed an alternative approach to locally or regionally deliver cytokines through GPI anchoring. Since the cytokines are anchored on the cell plasma membrane, they are immobilized so that a locally high concentration of cytokines could be achieved and their in vivo half-life could be elongated. Therefore, GPI anchored cytokines may be more effective than soluble versions. However, since GPI anchored proteins can release from the cells and are able to re-insert onto the plasma membrane of neighboring cells, this technique could help gene therapy by increasing gene delivery efficiency, as well as allowing GPI anchored proteins to be made in large scale and applied locally (such as to tumor masses) in high doses. One direct use of this technique is to inject GPI anchored IL-2 directly into tumor mass of cancer patients. Since the IL-2 upon injection will be anchored on regional tumor cells, a much higher dose could be used. Foreign MHC molecules and co-stimulatory molecules such CD80 or CD86 could be anchored onto tumor cells as well. Other potential uses of this technique include GPI anchorage of other cytokines or adhesion molecules on to tumor cells to mount immune responses against tumors. Therefore, GPI anchoring technology represents an interesting approach of cytokine-based immunotherapy.

A wide range of cell-surface proteins, including enzymes, coat proteins, surface antigens, and adhesion molecules, are attached to plasma membranes via GPI anchors. GPI anchors are also proposed to function in protein targeting, transmembrane signaling, and in the uptake of small molecules (endocytosis). GPI anchors of plasma membrane proteins are present in eukaryotes from protozoa and fungi to vertebrates (Doering, T. L. et al. (1990) J. Biol. Chem. 265:611-614; McConville, M. J. et al. (1993) Biochem. J. 294:305-324).

In one aspect, the invention, thus, entails attaching a GPI anchor to a factor which stimulates an immune response, thereby immobilizing the factor on the surface of a target cell. This strategy completely retains activity of the factor and represents an advance in the traditional approaches to immunotherapy because is substantially avoids systemic exposure and the resultant adverse reactions. In a preferred embodiment, a fusion gene was made by attaching a DNA oligo encoding human DAF GPI anchor signal in frame to the 3′ end of human IL-2 cDNA is described.

The present invention relates generally to compositions and methods for eliciting an immune response and enhancing current therapeutic modalities for the treatment of cancer. Specifically, the invention relates to making an immunogenic composition that is directed to a target cell surface and stimulates an immune response against the target cell.

The inventive methods are mediated by engineering immunomodulatory factors in a fashion so that they become attached to the surface of a target cell, typically a cancer cell. In different embodiments, this is accomplished either by delivering a nucleic acid molecule to a target cell and utilizing the cell's own protein expression machinery or by delivering the factor as a protein product directly to the cell. Once the factor is associated with the cell surface, an immune response is directed against the target cell.

Immunomodulatory Factors

Immunomodulatory factors embraced by the invention typically stimulate T cell proliferation. Preferably, the factor as described herein is a cytokine. As the skilled artisan will understand, certain interleukins and interferons are examples of cytokines that stimulate T cell proliferation.

In a preferred embodiment, the factor that stimulates T cell proliferation is IL-2. IL-2 is a product of activated T cells and supports T cell proliferation in an autocrine and paracrine manner. In other words, T cells stimulate their own proliferation by secreting IL-2 and synthesizing IL-2 cell surface receptors. In turn, IL-2 secreted by T lympocytes bind the IL-2 receptors and stimulate proliferation of activated T lymphocytes. IL-2 enhances non-specific immune responses such as natural killer (NK) and lymphokine-activated killer (LAK) cells. IL-2 also stimulates MHC-restricted cytotoxic T-cell responses.

IL-7 is also a preferred factor in the instant invention. IL-7 has also been shown to regulate T cells. IL-7 regulates B and T lymphocytes and has been shown to stimulate proliferation of cytolytic T cells and LAK cell in vitro and enhance their activities in vivo (Alderson et al., J Exp Med, 172:577 (1990)).

Still preferred, IL-12 is used in the instant invention. IL-12 is a heterodimeric immunoregulatory cytokine consisting of two disulfide-linked subunits p35 and p40. IL-12 is central to the initiation and maintenance of TH-1 type responses (Reiner et al., Curr Opin Immunol 7:360 (1995); Trinchieri et al., Leukoc Biol 59:505 (1996)). IL-12 functions as a growth factor of T and NK cells, promotes the development of T_{H}-1 type immune response, induces the secretion of interferon (IFN)-γ from resting and activated T and NK cells, and increases the cytotoxic activity of NK/lymphokine-activated killer (LAK) cells and specific cytotoxic T-lymphocyte (CTL) responses. In addition, IL-12 has been shown to present anti-angiogenic properties and have important anti-tumor and anti-metastatic effects against a number of murine tumors following administration by several different methods.

IL-15 and IL-18 are cytokines suitable for the instant invention. IL-15 can activate NK cells and stimulate proliferation of activated T cells and IL-18 was initially purified on its ability to promote IFN-γ production by T cells. IL-18 acts independently of IL-12 (Torigoe et al., J Biol Chem, 272:25737 (1997)) and synergistic effects of IL-12 and IL-18 on T cell IFN-γ production have been demonstrated (Micallef et al., Eur J Immunol, 26:1647 (1996)).

Interferons have also been noted for their ability to stimulate T cell proliferation. Interferons (IFN) were one of the first families of cytokines to be characterized in detail. Interferons can be divided into three classes: α, β and γ. IFN α and β are collectively known as type I interferons. Type I interferons stimulate NK cell activity and IFN-α can stimulate T_{H}-1 type T cell response (Belardelli et al., Immunol Today, 17:269 (1996)). Type I interferons also inhibit proliferation of certain cell types, including cancer cells. Id.

The instant invention also contemplates invention using the aforementioned cytokines in combination with one another. Accordingly, the factors of the instant invention can be used singly, or in combination with other factors that stimulate T cell proliferation.

Cell Surface Anchor

One salient feature of the invention is that the immunomodulatory factor is anchored to the surface of the target cell. In so anchoring the factor, the inventors believe that many adverse systemic effects can be avoided. Additionally, as demonstrated below in the Examples, anchored factors surprisingly elicit a stronger immune response than free factors.

In a preferred embodiment, factors used in the invention are attached to a target cell surface via a glycolipid linkage. Many cell surface proteins are anchored to the plasma membrane by a covalently attached glycolipid such as a glycosylphosphatidylinositol (GPI) anchor. Immediately following protein synthesis, a protein comprising a GPI modification signal is anchored to the ER lumen by a hydrophobic sequence 15-20 amino acids in length. Alberts et al., MOLECULAR BIOLOGY OF THE CELL, 3^{rd} Edition, p. 591 (1994). A GPI anchor is pre-assembled in the ER and following GPI attachment, the modified protein is glycosylated and shuttled to the exterior surface of the plasma membrane. GPI anchors are the preferred mode of attaching immunomodulatory factors to the surface of a target cell.

The process of covalently attaching a GPI anchor to the C-terminus of a peptide is catalyzed by enzymes in the rough ER. Enzymes of the ER cleave the original membrane-anchor sequence and attach a preassembled GPI intermediate to the freed protein. The anchor comprises a phosphoethanolamine (EthN-P), several sugars, including N-acetylglucosamine (GlcN) and mannose, linked to an inositol phospholipid. Because the proteins are attached to the cell surface only via their GPI anchors, they can be released in response to phospholipases such as phosphatidyl inositol-specific phospholipase C (PI-PLC).

Preferably, the GPI anchors of the present invention are mammalian. Mammalian GPI anchors typically comprise an oligosaccharide core consisting of GlcN, three mannose residues and a terminal EthN-P. A branching EthN-P is attached to the first mannose, and in a small population of GPI anchors, another branching EthN-P is attached to the second mannose. Medof et al., FASEB, 10:574-586 (1996).

Furthermore, the inositol phospholipid typically contains 1-alkyl, 2-acyl glyerol. The inositol phospholipids in anchors, however, can vary. For example, inositol phospholipids of proteins expressed on erythrocytes have an additional inositol-associated fatty acid that provides an additional point of attachment to the plasma membrane. Id. Such anchors are described as being “two footed.” Accordingly, the GPI anchors of to the present invention can be “one footed,” “two footed” or “three footed,” as described above.

The GPI anchors suitable for the present invention also includes those described in Bandman, et al., U.S. Pat. No. 5,968,742, as well as other GPI anchor analogs and derivatives thereof. As used generally in the art, “derivative” refers to a compound obtained from another compound by a simple chemical process; e.g., acetic acid is a derivative of alcohol. An “analog” is a compound that shares a common structural feature with its base compound, but is not necessarily derived from it. In a preferred embodiment, a GPI sequence of decay accelerating factor (DAF) is used.

Nucleic Acids

In addition to the GPI-linked factors described above as proteins, the invention also contemplates the nucleic acids encoding those proteins. Preferably, the factor is encoded in a vector, like a plasmid or a viral vector. Also preferred, the factor encoded by the nucleic acid is a cytokine, and still preferred, the cytokine is IL-2 or IL-12. The DNA and protein sequences for a preferred GPI-linked IL-2 are presented in SEQ ID NOS: 1 and 2.

The nucleic acid used in the invention can be substantially any nucleic acid encoding a factor that stimulates T cell proliferation. The length of the nucleic acid is not critical to the invention. Any number of base pairs up to the full-length gene may be transfected. For example, the nucleic acid can be a linear or circular double-stranded DNA molecule having a length from about 100 to 10,000 base pairs in length, although both longer and shorter nucleic acids can be used.

The nucleic acid can be DNA. For example, linear or circular and can be single- or double-stranded. DNA includes cDNA, triple helical, supercoiled, Z-DNA and other unusual forms of DNA, polynucleotide analogs, antisense DNA, DNA encoding a portion of the genome of an organism, gene fragments, and the like.

The nucleic acid can also be RNA. For example, antisense RNA, catalytic RNA, catalytic RNA/protein complex (i.e., a “ribozyme”), a viral genome fragments such as viral RNA, RNA encoding a protein such as a therapeutic protein and the like. The nucleic acid can be selected on the basis of a known, anticipated, or expected biological activity that the nucleic acid will exhibit upon delivery to the interior of a target cell or its nucleus.

The nucleic acid can be prepared or isolated by any conventional means typically used to prepare or isolate nucleic acids. For example, DNA and RNA molecules can be chemically synthesized using commercially available reagents and synthesizers by methods that are described, for example, by Gait, 1985, in OLIGONUCLEOTIDE SYNTHESIS: A PRACTICAL APPROACH (IRL Press, Oxford). RNA molecules also can be produced in high yield via in vitro transcription methods using plasmids such as SP65, which is available from Promega Corporation (Madison, Wis.). The nucleic acid can be purified by any suitable means; many such means are known in the art. For example, the nucleic acid can be purified by reverse-phase or ion exchange HPLC, size exclusion chromatography, or gel electrophoresis. Of course, the skilled artisan will recognize that the method of purification will depend in part on the size of the DNA to be purified. The nucleic acid can also be prepared using any of the innumerable recombinant methods which are known or are hereafter developed.

The nucleic acid encoding one or more proteins of interest can be operatively associated with a variety of different promoter/regulator sequences. The promoter/regulator sequences can include a constitutive or inducible promoter, and can be used under the appropriate conditions to direct high level or regulated expression of the gene of interest. Particular examples of promoter/regulatory regions that can be used include the cytomegalovirus promoter/regulatory region and the promoter/regulatory regions associated with the SV40 early genes or the SV40 late genes. Substantially any promoter/regulatory region which directs high level or regulated expression of the gene of interest can be used.

The nucleic acid described herein can be recombinantly engineered into a variety of known host vector systems that provide for replication of the nucleic acid. These vectors can be designed, using known methods, to contain the elements necessary for directing transcription, translation, or both, of the nucleic acid in a cell to which it is delivered. Methods which are known to the skilled artisan can be used to construct expression constructs having the protein coding sequence operably linked with appropriate transcriptional/translational control signals. These methods include in vitro recombinant DNA techniques and synthetic techniques. For example, see Sambrook et al., 1989, MOLECULAR CLONING: A LABORATORY MANUAL, Cold Spring Harbor Laboratory (New York); Ausubel et al., 1997, CURRENT PROTOCOLS IN MOLECULAR BIOLOGY, John Wiley & Sons (New York).

Vector systems can be viral or non-viral. Particular examples of viral vector systems include adenovirus, retrovirus, adeno-associated virus and herpes simplex virus. Preferably, an adenovirus vector is used. An example of a non-viral vector system is a plasmid. Preferably, the vector is a plasmid. Among the viral vectors, conditionally replicating adenoviruses are preferred, and especially preferred are those adenoviruses which replicate selectively in p53 deficient cells.

The vector containing a nucleic acid as described herein can be an expression construct that comprises DNA encoding a protein, a transcribable construct comprising DNA encoding ribozymes or antisense RNA, expression constructs comprising RNA that can be directly translated to generate a protein product, or that can be reverse transcribed and either transcribed or transcribed and translated to generate an RNA or protein product, respectively, and transcribable constructs comprising RNA having any promoter/regulatory sequence necessary to enable generation of DNA by reverse transcription.

Pharmaceutical Compositions

In a related vein, this invention also contemplates a pharmaceutical composition comprising the proteins and/or nucleic acids of the present invention and a pharmaceutically suitable excipient. For example, the pharmaceutical composition of the instant invention comprises an immunogenic composition comprising a factor that stimulates T cell proliferation and a GPI anchor and a pharmaceutically suitable excipient. Also preferred, the pharmaceutical composition further comprises a cancer cell. Preferably, the cancer cell is a melanoma cell.

Such a pharmaceutical composition can comprise one or more pharmaceutically suitable excipients, one or more additional ingredients, or some combination of these. Local or regional treatment instead of systemic application of the pharmaceutical composition of the instant invention is also contemplated. This is an alternative approach to getting high therapeutic effect and low toxic side effects.

For oral administration, the pharmaceutical compositions can take the form of, for example, tablets or capsules prepared by conventional means with pharmaceutically suitable excipients such as binding agents (e.g., gelatin, acacia, pregelatinized maize starch, polyvinylpyrrolidone and hydroxypropyl methylcellulose); fillers (calcium carbonate, sodium carbonate, lactose, microcrystalline cellulose, calcium phosphate, calcium hydrogen phosphate and sodium phosphate); lubricants (e.g., magnesium stearate, stearic acid, silica and talc); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically suitable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds formulated herein are for local administration by injection (i.e., intralesional). Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. Subcutaneous, intramuscular and intravenous injections may also be considered local administration, depending on the location of the target cell intended for treatment.

The compounds can also be formulated in rectal compositions such as suppositories or retention enemas, e.g., containing conventional suppository bases such as cocoa butter or other glycerides.

Douche preparations or suspensions for vaginal irrigation can be made by combining the composition described herein, with a pharmaceutically acceptable liquid carrier. As is known in the art, douche preparations can be administered using, and can be packaged within, a delivery device adapted to the vaginal anatomy of the subject. Douche preparations can further comprise various additional ingredients, including antioxidants, antibiotics, antifungal agents, and preservatives.

Vaginal preparations of the composition described herein can also be used for administration in utero of the nucleic acid described herein to an ovum, embryo, fetus, or a neonate during birth. Such preparations are preferably placed in the uterus of the woman bearing the ovum, embryo, fetus, or neonate, although such preparations can also be placed cervically or vaginally, or can be physically contacted with the embryo or fetus or on or within the chorionic or amniotic membranes.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions is contemplated include humans, primates and other mammals.

Methods

The invention further contemplates various methods of preparing therapeutic molecules and pharmaceutical compositions, as well as therapeutic methods.

For example, the instant invention provides for a method of making an immunogenic nucleic acid composition. Typically such a composition will contain a vector and comprises modifying a nucleic acid encoding a factor that stimulates T cell proliferation such that it includes a sequence that signals a host cell to attach a GPI anchor. Preferably, the nucleic acid encodes a cytokine that stimulates T cell proliferation. Still preferred, the cytokine is IL-2 or IL-12.

Also contemplated in the present invention is a method of treating a patient. It is believed that such a therapeutic method will elicit an immune response against a target cell, which will have a beneficial therapeutic effect on the patient. Generally, this method involves administering to a patient a therapeutically effective amount of a pharmaceutical composition, as described above. A typical composition will contain a vector comprising (a) a nucleic acid encoding a factor that stimulates T cell proliferation attached to a sequence that signals a GPI anchor and (b) a pharmaceutically suitable excipient.

In one preferred method, target cells are treated with a therapeutic composition ex vivo, and then administered back to the patient, as a sort-of vaccine. In one embodiment, this method entails transferring a nucleic acid encoding a GPI-linked factor into a target cell. This utilizes a the protein production machinery of the target cell to produce the GPI-linked product. The target cells then are administered to the patient as vaccine. This method is illustrated by the Examples. Preferred target cells are cancer cells. The target cell also may be a hybrid cell, such as a hybrid between a cancer cell and an antigen presenting cell, like a dendritic cell.

In another ex vivo embodiment, target cells are coated or “painted” with the GPI-linked factor. This may be accomplished in several different ways. First, the factor may be produced by recombinant means, isolated and used to coat target cells. Because of the lipid tail on the GPI moiety, the factor will become associated with the target cell. In a preferred aspect, however, the target cells are coated using “feeder cells.” In this aspect, cells expressing the GPI-linked factor are maintained in culture. These feeder cells are co-cultured with the target cells, with an intervening membrane which allows GPI-factor but not cells to pass through it. This aspect takes advantage of the observation that cells producing GPI-linked protein shed the GPI protein with some frequency and that protein may become associated with other cells. Again, in both of these aspects, the coated target cells may be administered to a patient as a vaccine. Additionally, in the case where the target cells are cancer cells (or hybrids of cancer cells) and such an ex vivo approach is used, it may be beneficial to irradiate the cells prior to administering them to a patient.

In addition to in vivo applications, cell surface engineering using GPI anchored proteins has several advantages to traditional gene transfer approaches in vitro: (i) the method is applicable to cells that are difficult to transfect, (ii) the method can be used when only a small number of cells are available or when cells cannot be easily propagated, (iii) the cell surface can be modified irrespective of cell type, (iv) the amount of the protein ultimately displayed on the cell surface can be precisely controlled and (v) multiple GPI-anchored proteins can be incorporated sequentially or simultaneously into the same cells (Medof et al., FASEB J, 10:574 (1996)). Accordingly, the immunogenic composition and methods described in the present application can be used in vitro, to enhance transfection of cells with IL-2.

The invention is now described with reference to the following examples, which are provided for illustration only. The invention is not limited to the examples, but rather includes all variations which are evident as a result of the teaching provided therein.

### EXAMPLE 1

### GPI Anchored IL-2 and IL-12 Expression Vectors

Human IL-2 cDNA was inserted into pcDNA3.1(+) expression plasmid between KpnI/XbaI site. The stop codon was removed by PCR and the GPI anchor sequence was synthesized according to human DAF cDNA sequence and in frame insert into downstream of IL-2 3′ end.

Preferably, the DAF sequence is inserted first. A DNA fragment (114 bp) encoding the GPI anchor signal for DAF was generated by annealing two synthesized, complimentary DNA oligos with XhoI site at the 5′ end and XbaI site at the 3′ end and inserted into plasmid pcDNA3.1. Human IL-2 cDNA without the stop codon was generated by PCR from IL-2 cDNA (ATCC 59396) using primers 5′ GGGGTACCTAATCACTACTCACAGTAAC 3′ and 5′ CCGCTCGAGAGTTAGTGTTGAGATGATGC 3′, which was then inserted in front of the DAF fragment in frame, resulting in plasmid pcDNA3.1-IL2gpi. The entire sequence of the fusion gene was confirmed by an automatic DNA sequencer (Perkin Elmer 310). As a control vector, pcDNA3.1-sIL2 encoding a secreted form of IL-2 was generated as well.

The murine IL-12 B (p40) cDNA subunit was inserted into pcDNA3.1(+)/Zeo expression plasmid between EcoRI/XhoI sites. The stop codon was removed by PCR and the GPI anchor sequence was inserted in frame into the same plasmid between XhoI and XbaI. Additionally, murine IL-12 A (p35) cDNA was inserted into another pcDNA3.1(+)/Zeo plasmid between EcoRI/Xho sites with stop codon on it. The entire fragment of p35 and the pCMV promoter was cut off by BglII/StuI and blunt ended with Klenow. This fragment was inserted into pcDNA3.1(+)/Zeo-IL12-B-GPI plasmid which was cut with BglII and blunt ended with Klenow. The new recombinant plasmid was named pIL12-A-BGPI and contains both the A and B subunit of IL-12, in which A is secreted and B is linked with a GPI anchor sequence.

### EXAMPLE 2

### Transfection and Selection of Stable Cell Lines

B16F0 murine melanoma cells were purchased from American Type Culture Collection (CRL-6322) and maintained on DMEM medium containing 10% v/v fetal bovine serum (FBS, HyClone, Logan, Utah) and gentamycin (Gibco BRL, Grand Island, N.Y.). IL-12 responsive T cell clone 2D6 was maintained in RPMI 1640 medium containing 10% v/v FBS, rIL-12 (250 pg/ml), 5×10⁵ 2-mercapto-ethanol and gentamycin.

### Example 2.1

B16F0 murine melanoma cells were transfected with pcDNA3.1-IL2-GPI (G418 resistant; encoding IL-2-GPI) only, pIL2-A-BGPI (Zeocin resistant; encoding IL-12-GPI) only or both pcDNA3.1-IL2-GPI and pIL12-A-BGPI, and Lipofectamine (Life Technologies) transfection reagent. The stable cell lines were selected in DMEM media containing 1000 mg/ml G418, 500 mg/ml Zeocin or both, as appropriate, for 3-4 weeks.

### Example 2.2

In another study, pcDNA3.1-IL2gpi and pcDNA3.1-sIL2 were transfected into B16F0 tumor cells using Lipofectamine transfection reagents according to the manufacturer's protocol (Gibco BRL). Stable cell lines were selected from DMEM containing 1000 μg/ml G418 (Sigma, St. Louis, Mo.) for 3-4 weeks and named B16F0/IL2s for cells expressing secreted IL-2 and B16F0/IL2gpi for cells expressing GPI anchored IL-2.

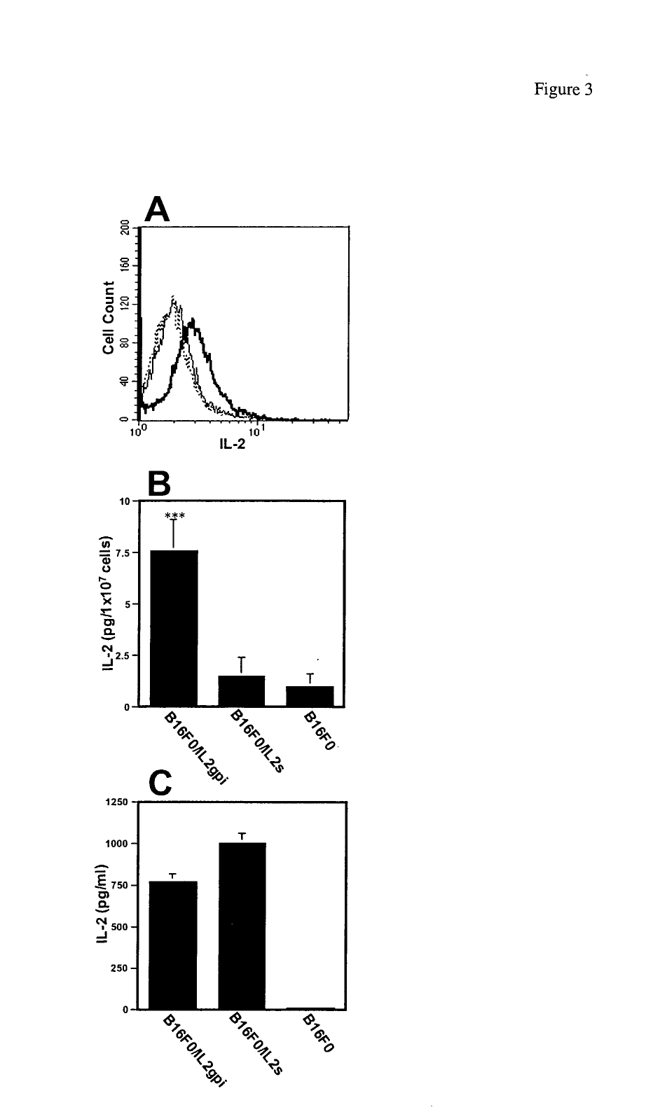

FACS analysis. In order to determine if GPI anchored IL-2 is expressed on the cell surface, 1×10⁶ B16F0/IL2gpi cells were stained with anti-human IL-2-FITC and analyzed using the FACS Calibur (Backton/Dikinson, San Jose, Calif.). Only B16F0/IL2gpi cells showed a significant amount of IL-2 on their cell surface. To further determine whether the plasma membrane binding of IL-2 is through the GPI anchor, 1×10⁶ B16F0/IL2gpi cells were treated with PI-PLC, which cleaves protein from the lipid anchor. After PI-PLC treatment, there was no detectable IL-2 on the cell surface (FIG. 3A). To measure the absolute amount of GPI anchored IL-2 on the cell surface, 1×10⁷ B16F0 cells, B16F0/IL2s cells, and B16F0/IL2gpi cells were treated with PI-PLC. The amount of IL-2 in the supernatants was assayed by ELISA. As shown in FIG. 3B, B16F0/IL2gpi cells expressed significantly increased amounts of IL-2 on the cell surface compared to B16F0 cells or B16F0/IL2s cells. In addition, IL-2 was detected in the culture medium of B16F0/IL2gpi cells, indicating GPI anchored IL-2 was released from the cells (FIG. 3C). After co-culture of B16F0 cells with B16F0/IL2gpi cells in a unique culture system, in which two different types of cells can be co-cultured without physical contact but allows growth factors to be shared by both cells, IL-2 activity was detected on the B16F0 cell surface, confirming that the released IL-2 from B16F0/IL2gpi cells is still in the GPI anchored form and able to re-coat the plasma membrane (data not shown).

ELISA assay. To quantitatively measure the amount of GPI anchored IL-2 on the cell surface, 1×10⁷ B16F0 cells, B16F0/IL2s cells, and B16F0/IL2gpi cells were harvested. After two washings with PBS, each cell pellet was dissolved with 0.2 ml of PI-PLC solution (8 U/ml, Sigma) and incubated at 37° C. for one hour. The supernatants were collected and the amount of IL-2 in them was measured with the QuantiGlo ELISA kit (R&D Systems, Minneapolis, Minn.). The same experiment was repeated three times and the results were reported as an average.

### EXAMPLE 3

### Animal Testing

C57BL/6J female mice were purchased from the Jackson Laboratory at the age of 4-6 weeks. All experiments were performed according to the National Institutes of Health guidelines for care and use of laboratory animals.

### Example 3.1

### Tumor Cell Infiltration Assay

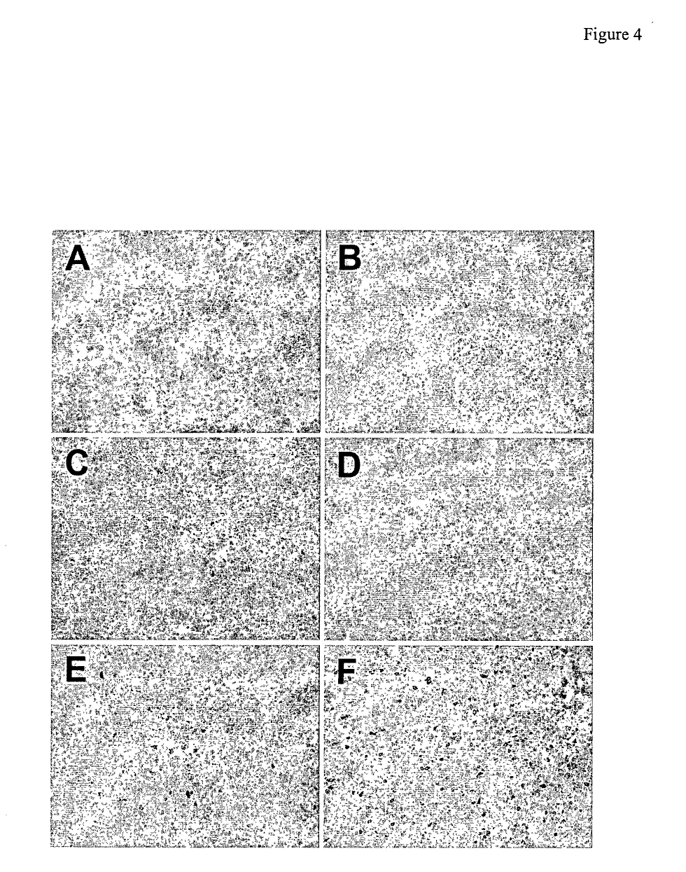

In order to test whether the GPI anchored IL-2 still keeps its biological function, B16F0 cells, B16F0/IL2s cells or B16F0/IL2gpi cells were subcutaneously injected into mice and ten days later the tumors were recovered. Lymphocytes infiltrated into the tumors were detected by immunohistochemical assay. The results demonstrated that the expression of GPI anchored IL-2 significantly increased T lymphocyte infiltration in tumors (FIGS. 4A-4F) while the secreted IL-2 in this situation did not show any function.

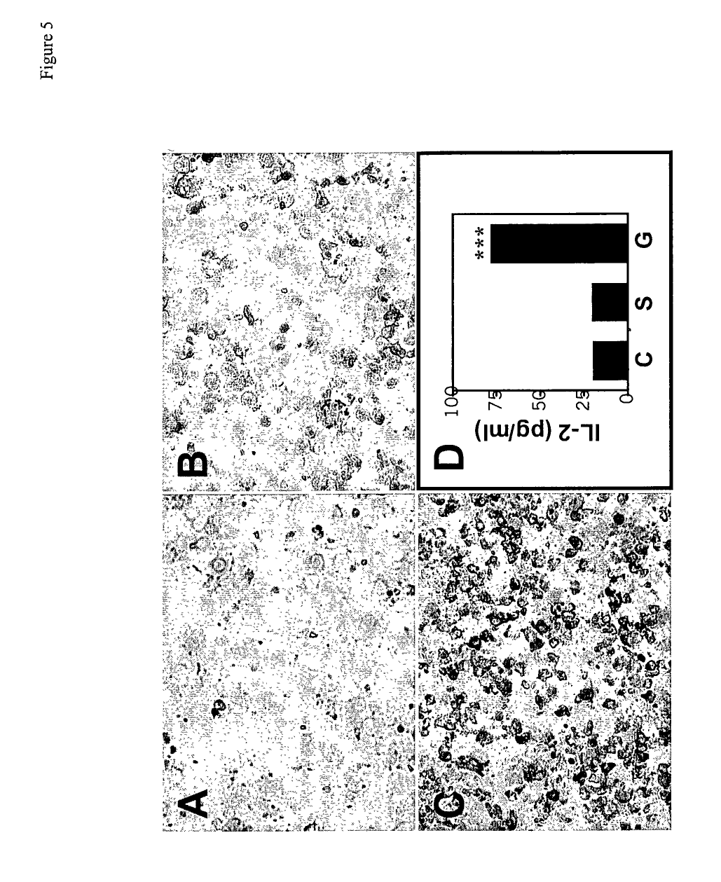

Female C57BL/6J mice were each subcutaneously injected with 1×10⁵ B16F0 cells, B16F0/IL2s cells, or B16F0/II2gpi cells. Local high level of IL-2 achieved by GPI anchored IL-2. In order to determine if local high dose of IL-2 can be achieved by GPI anchored IL-2, B16F0 cells, B16F0/IL2s cells or B16F0/IL2gpi cells were subcutaneously injected into C56BL/6J mice. Two weeks later the tumors were excised and tumor cells were isolated. Tumor cell slides were produced by Cytospin. The cells were then stained for cell surface IL-2 by immunohistochemistry. Only tumor cells that express GPI anchored IL-2 showed significantly high levels of IL-2 compared to tumor cells that express secreted IL-2 or regular B16F0 cells (FIGS. 5A-5D). In order to be certain that the cell surface IL-2 on cells from B16F0gpi tumor is in the GPI anchored form, tumor cells from experimental tumors, after washing with PBS, were treated with PI-PLC. IL-2 in the supernatant was measured by ELISA. Only the cells isolated from B16F0gpi tumor generated significantly high levels of IL-2 (FIG. 5D).

### Example 3.2

### In vivo Tumor Study

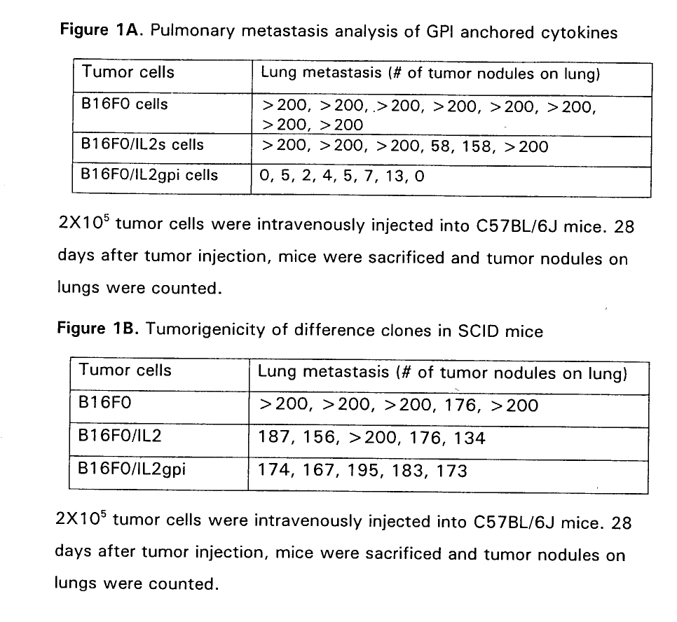

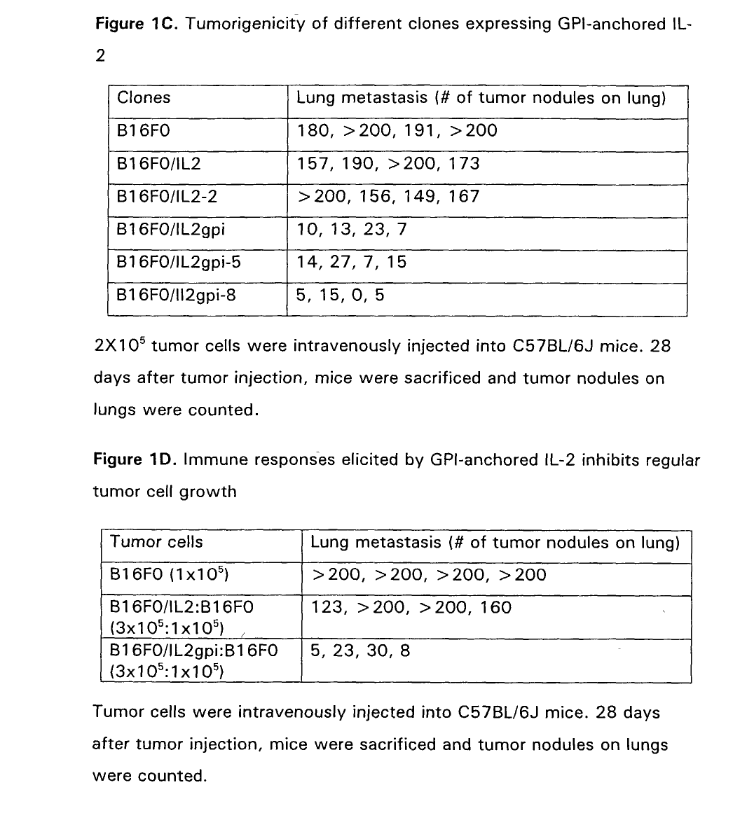

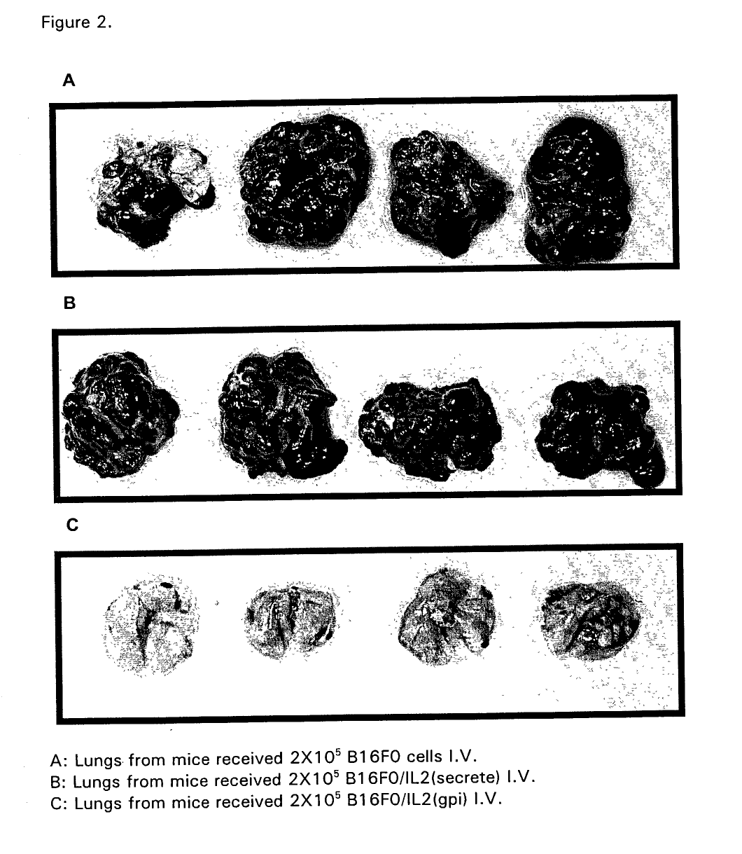

Three groups of female C57bl/6J mice (four per group) were injected intravenously with 2×10⁵ cells in 40 μl PBS as follows: B16F0 cells alone (control), B16F0 cells expressing IL-2-gpi (B16F0/IL2gpi), and (5) B16F0 cells expressing soluble IL-2 (B16F0/IL2s). Mice were killed on day 23, the lungs were excised and tumor nodules on the lungs were counted. These studies show that GPI anchored IL-2 retain their biological activity when transfected in host cells and that GPI anchored cytokines are more effective in promoting a host immune response than soluble IL-2 (FIGS. 1A-1D and 2A-2C).

The data were summarized in FIG. 1A from two independent experiments and photographs were taken from one experiment (FIGS. 2A-2C). GPI anchored IL-2 dramatically inhibited tumor growth, while secreted IL-2, although expressed at the same level (FIG. 3C), did not show any anti-tumor effect. In order to rule out the possibility that this tumor growth inhibition effect is due to the variable levels of tumorigenicity of individual clones independent on any immune responses elicited, two experiments were performed. First, the tumorigenicity of the three clones, B16F0, B16F0/IL2 and B16F0IL2gpi, was tested in immunodeficient mice (SCID) by pulmonary metastasis assay. The results showed that there is no significant difference in tumorigenicity among the three clones (FIG. 1B). Second, two more stable clones that express GPI anchored IL-2 were selected and tested. Similar results were obtained (FIG. 1C).

To determine whether the immune responses elicited by GPI-anchored IL-2 can inhibit regular B16F0 cell growth, the following experiment was performed. 1×10⁵ B16F0 cells were mixed with 3×10⁵ of B16F0/IL2 cells and B16F0/IL2gpi cells, respectively, and intravenously injected into mice (4 for each group). In the control group, only 1×10⁵ B16F0 cells/mouse were injected. Four weeks later the lung nodules were counted. The results (FIG. 1D) showed that immune responses generated by GPI-anchored IL-2 are effective to regular tumor cells.

To confirm GPI anchored cytokines were expressed on the cell membrane, phosphatidylinositol specific phospholipase C (PI-PLC) was used to digest the cell surface membrane anchored proteins. The cells were collected and washed twice in phosphate buffered saline (PBS), treated with PI-PLC (8 μg/ml) at 37° C. for 1 hour. Cells were then spun down and the supernatant was collected for the ELISA assay. Human IL-2 and murine IL-12 ELISA kits were purchased commercially. (R&D Research). This assay showed a local high dose IL-2 was achieved on the surface of tumor cells transfected with pcDNA3.1-IL-2-GPI.

As a measure of the immune response elicited against the tumor cells, two mice were treated as above and sacrificed at day 14. The tumors were removed and homogenized, and then assayed for the presence of T-cells using antibodies to CD3 and CD19. Result indiated that the number of tumor infiltrating lymphocytes (TIL) had significantly increased in the GPI-IL-2 transfected tumor cells compared with untransfected tumor cells.

| | |
|---|---|
| 5′ CCA AAT AAA GGA AGT GGA ACC ACT TCA | SEQ ID NO 1 |
| GGT ACT ACC CGT CTT CTA TCT GGG CAC ACG | |
| TGT TTC ACG TTG ACA GGT TTG CTT GGG ACG | |
| CTA GTA ACC ATG GGC TTG CTG ACT 3′.: | |
| PNKGS GTTSG TTRLL SGHTC FTLTG LLGTL | SEQ ID NO 2 |
| VTMGL LT | |

What is claimed is:

1. An immunogenic composition containing a vector comprising a nucleic acid encoding a factor that stimulates T cell proliferation attached to a sequence that signals a GPI anchor.

2. The immunogenic composition of claim 1, wherein said vector is a plasmid or a virus vector.

3. The immunogenic composition of claim 1, wherein said factor that stimulates T cell proliferation is a cytokine.

4. The immunogenic composition of claim 3, wherein said cytokine is IL-2.

5. The immunogenic composition of claim 3, wherein said cytokine is IL-12.

6. A pharmaceutical composition, comprising the immunogenic composition of claim 1 and a pharmaceutically suitable excipient.

7. The pharmaceutical composition of claim 6, wherein said vector is a plasmid or a virus vector.

8. The pharmaceutical composition of claim 6, wherein said factor that stimulates T cell proliferation is a cytokine.

9. The pharmaceutical composition of claim 8, wherein said cytokine is IL-2.

10. The pharmaceutical composition of claim 8, wherein said cytokine is IL-12.

11. A method of making an immunogenic composition containing a vector comprising (i) modifying a nucleic acid encoding a factor that stimulates T cell proliferation to include a sequence that signals a GPI anchor.

12. The method of making of claim 11, wherein said vector is a plasmid or a virus vector.

13. The method of making of claim 11, wherein said factor that stimulates T cell proliferation is a cytokine.

14. The method of making of claim 13, wherein said cytokine is IL-2.

15. The method of making of claim 13, wherein said cytokine is IL-12.

16. A method of eliciting an immunogenic response, comprising (i) contacting a target cell with an immunogenic composition containing a vector comprising (a) a nucleic acid encoding a factor that stimulates T cell proliferation attached to a sequence that signals a GPI anchor.

17. The method of eliciting an immunogenic response of claim 16, wherein said vector is a plasmid or a virus vector.

18. The method of eliciting an immunogenic response of claim 16, wherein said factor that stimulates T cell proliferation is a cytokine.

19. The method of eliciting an immunogenic response of claim 18, wherein said cytokine is IL-2.

20. The method of eliciting an immunogenic response of claim 18, wherein said cytokine is IL-12.

21. The method of eliciting an immunogenic response of claim 16, wherein said target cell is a cancer cell.

22. The method of eliciting an immunogenic response of claim 21, wherein said cancer cell is a melanoma cell.

23. A method of treating a patient comprising (i) administering a therapeutically effective amount of the pharmaceutical composition of claim 6.

24. The method of treating of claim 23, wherein said vector is a plasmid or a virus vector.

25. The method of treating of claim 23, wherein said factor that stimulates T cell proliferation is a cytokine.

26. The method of treating of claim 25, wherein said cytokine is IL-2.

27. The method of treating of claim 25, wherein said cytokine is IL-12.

28. An immunogenic composition comprising a factor that stimulates T cell proliferation and a GPI anchor.

29. The immunogenic composition of claim 28, wherein said factor is a cytokine.

30. The immunogenic composition of claim 29, wherein said cytokine is IL-2.

31. The immunogenic composition of claim 29, wherein said cytokine is IL-12.

32. A pharmaceutical composition, comprising the immunogenic composition of claim 28, further comprising a pharmaceutically suitable excipient.

33. A method for preparing a cancer vaccine comprising (i) preparing a feeder layer of cells that express a factor that stimulates T cell proliferation on their plasma membrane, (ii) exposing a cancer cell or cancer cell hybrid to said feeder layer, (iii) optionally irradiating said cancer cell or said hybrid and (iv) administering said exposed cancer cell or said hybrid to a patient.

34. The method of claim 33, wherein said factor is a cytokine.

35. The method of claim 34, wherein said cytokine is IL-2.

36. The method of claim 34, wherein said hybrid cell is a fusion between a cancer cell and a dendritic cell.

37. The method of claim 33, wherein said cancer cell is a melanoma cell.

38. An immunogenic composition comprising a factor that stimulates T cell proliferation attached to the plasma membrane of a cell via a GPI anchor, wherein said cell is a cancer cell.

39. The immunogenic composition of claim 38, wherein said factor is a cytokine.

40. The immunogenic composition of claim 39, wherein said cytokine is IL-2.

41. The immunogenic composition of claim 39, wherein said cytokine is IL-12.

42. The immunogenic composition of claim 38, wherein said cancer cell is a melanoma cell.

43. The method of claim 2, wherein said virus is a conditionally replicating adenovirus.

44. The method of claim 7, wherein said virus is a conditionally replicating adenovirus.

45. The method of claim 12, wherein said virus is a conditionally replicating adenovirus.

46. The method of claim 17, wherein said virus is a conditionally replicating adenovirus.

47. The method of claim 24, wherein said virus is a conditionally replicating adenovirus.
